(19) United States
(12) Patent Application Publication
(10) Pub. No.: US 2004/0259744 A1
(43) Pub. Date: Dec. 23, 2004

(54) Skin and hair cleansers containing sulfur

(76) Inventors: Meidong Yang, Santa Rosa, CA (US); Gordon J. Dow, Santa Rosa, CA (US)

Correspondence Address: HOWARD EISENBERG, ESQ., 2206 APPLEWOOD COURT, PERKASIE, PA, 18944, US

(21) Appl. No.: 10/742,321

(22) Filed: Dec. 19, 2003

(51) Int. Cl. A61K 7/075

(52) U.S. Cl. 510/127

(57) ABSTRACT

A sulfur-containing cleanser formulation containing a surfactant and a lipophilic emulsifying agent at a concentration of 7 w/w % or greater. The cleanser formulation has reduced sulfur malodor compared to a similar formulation with a concentration of lipophilic emulsifying agent less than 7 w/w %.

### FIELD OF THE INVENTION

The invention pertains to the field of topical cleanser used for the treatment of disorders of the skin. Particularly, the invention pertains to skin cleansers and shampoos containing sulfur.

### BACKGROUND OF THE INVENTION

The combination of sulfur and a sulfonamide, such as sodium sulfacetamide, has been successfully utilized in the treatment of skin disorders, such as acne vulgaris, rosacea, and seborrheic dermatitis. Sulfonamides have antibacterial activity and sulfur is thought to be keratolytic and keratoplastic although the precise mechanism of action of sulfur is not known. The combination of sulfur and a sulfonamide applied topically has been reported to be of benefit in treating acne vulgaris, rosacea, and seborrheic dermatitis. A common combination of sulfonamide and sulfur is a formulation containing 10 w/w % sodium sulfacetamide and 5 w/w % sulfur.

A significant problem exists with such topical sulfur-containing formulations, particularly those intended for use on the head, especially on the scalp and face. Such formulations have an intrinsic malodor, especially when applied to the skin, which results in decreased patient acceptance and reduced patient compliance. Typically, the malodor of such products lingers for one to several hours and is noticeable to the user, especially when applied to the face or scalp, due to the proximity to the nose, thus discouraging patients from using such medicated topical products as often as prescribed. Poor patient compliance can adversely affect the effectiveness of a treatment regimen.

A variety of techniques have been utilized to attempt to reduce or eliminate the odor associated with sulfur-containing formulations. Schacknai et al, WO 02/02059, discloses that sulfur-containing formulations having a pH between 6.0 to 8.5 have reduced malodor compared with formulations having a pH outside of this range. Schacknai further discloses that malodor of sulfur-containing formulations increases as the pH is increased above about 7.7 or decreased below about 7.0. Thus, as disclosed in Schacknai, sulfur malodor may be reduced by formulating a sulfur-containing composition to have a pH between 6.0 and 8.5, and preferably between 7.0 and 7.5.

Most commonly, the malodor associated with sulfur is masked by the inclusion of a fragrance in sulfur-containing formulations. It is noted that a fragrance is included in each one of the sulfur-containing compositions disclosed in Schacknai.

The inclusion of fragrances in a dermatologic formulation, such as a sulfur-containing formulation, is disadvantageous because many individuals suffer deleterious reactions upon exposure to fragrances. Allergic and irritation reactions to topically applied formulations are frequently encountered with the use of fragrances contained in such formulations. Moreover, fragrance-free products permit a user to apply his or her own perfume, cologne, after-shave, or other fragrance-containing cosmetic concurrently with the sulfur-containing formulation. Therefore, it is desirable to eliminate fragrances from topical sulfur-containing skin formulations if possible.

A sodium sulfacetamide 10% and sulfur 5% lotion is marketed by Hope Pharmaceuticals (Scottsdale, Ariz.). This lotion also contains propylene glycol, isopropyl myristate, propylene glycol monostearate, cetyl alcohol, PEG-8 stearate, benzyl alcohol, sodium thiosulfate, edetate sodium, monobasic sodium phosphate, emulsifying wax, and purified water. The Hope Pharmaceuticals lotion product is similar to NOVACET® (GenDerm Corp., Lincolnshire, Ill.).

These lotions are used to treat topical conditions such as acne vulgaris, rosacea, and seborrheic dermatitis by applying a thin film of the lotion to affected areas one or more times per day. These lotions are left on the skin after application and are not suitable as cleansers. In order for a composition to be useful as a skin cleanser or shampoo, it must contain one or more surfactants in sufficient concentration to clean the skin or hair. Such surfactants are more specifically known as detergents for their cleansing properties and are highly hydrophilic, that is they have a relatively high HLB value. Generally, skin cleansers contain surfactants at a total concentration of between 5% and 50%.

Such a skin cleanser with sulfur and sodium sulfacetamide is PLEXION™ Cleanser (Medicis Pharmaceutical Corp., Scottsdale, Ariz.). This product retains a distinct and unpleasant sulfur malodor, despite the fact that it is the commercial product described by Schacknai (WO 02/-2059), which is disclosed as having a reduced malodor during storage based on its pH. Although Schacknai has disclosed that the malodor of sulfur-containing preparations is reduced when formulated at a pH of between 6.0 and 8.5, it is the experience of the inventors that such products do not address the problem of residual malodor on the patient's skin for one or more hours following use. Such malodor is particularly troublesome for a skin cleanser product because the malodor lingers on the skin of the user. Other than using fragrances to mask the sulfur malodor, to date, no solution has been found for the problem of sulfur malodor in sulfur-containing cleanser and shampoo formulations.

### SUMMARY OF THE INVENTION

It has been unexpectedly discovered that the inclusion in a sulfur-containing cleanser formulation of a lipophilic emulsifying agent, such as emulsifying wax, at a concentration of 7% or higher significantly reduces the malodor, especially the residual malodor, typically associated with the sulfur when applied to the skin or hair of a human or animal patient. The sulfur malodor is especially a problem with skin cleanser or shampoo formulations which are washed off the body after applying. With these formulations, excipients in the formulation which may help control the malodor are washed off and are unavailable to help combat sulfur malodor after application. This is in contrast to “leave-on” sulfur-containing formulations, such as lotions, which are not washed off and which therefore remain on the skin for extended periods. Such formulations retain on the skin the various combination of excipients that help to control sulfur malodor. Thus, the presence or absence of various ingredients of “leave-on” sulfur-containing products is not pertinent to the composition of “wash-off” products such as skin cleansers or shampoos. It is noted that there are no presently marketed sulfur-containing cleanser products that do not contain a fragrance.

The term “residual odor” refers to the malodor associated with sulfur-containing cleanser products that remains after the cleanser has been washed from the skin or hair, and especially that which remains more than one hour after the cleanser has been washed from the skin or hair.

As described below, the most preferred concentration of lipophilic emulsifier in the sulfur-containing cleanser formulation of the invention is about 15%. Concentrations of lipophilic emulsifier above or below 15% may be used if desired. However, little additional malodor reducing benefit appears to be obtained from concentrations of lipophilic emulsifier above 15% in the formulation containing the common range of surfactant concentration.

In this specification and in the claims, the term “cleanser” is a generic term that refers to either a skin cleanser or a shampoo or to a cleanser that is both a skin cleanser and a shampoo. The term “skin cleanser” is a specific term that refers to a cleanser that is used primarily for the skin. The term “shampoo” is a specific term that refers to a cleanser that is used primarily for the hair.

In this specification, the invention is described with reference to a sulfur-containing cleanser formulation containing a sulfonamide, in particular sulfacetamide. It is to be understood, however, that the sulfur-containing skin cleanser or shampoo formulation containing a sulfonamide, and particularly sulfacetamide is merely illustrative of the invention. The invention pertains to any sulfur-containing cleanser formulation, whether or not the formulation contains a sulfonamide. Further, the invention pertains to any sulfur-containing cleanser formulation containing a sulfonamide, and sulfacetamide is merely illustrative of sulfonamides that may be included in the formulation of the invention. Additionally, emulsifying wax is used herein as an illustration of a lipophilic emulsifier. One skilled in the art will understand that other lipophilic emulsifying agents may be used in accordance with the invention. All concentrations described herein are w/w.

### DETAILED DESCRIPTION OF THE INVENTION

In a first embodiment, the invention is a sulfur-containing cleanser formulation containing one or more surfactants and a lipophilic emulsifying agent at a concentration of about 7% or higher. Most preferably, the concentration of the lipophilic emulsifying agent is about 15%. Concentrations between 7% to 15% or higher are also suitable for the invention. Preferably, the concentration of lipophilic emulsifying agent is 8% or higher, more preferably 10% or higher, even more preferably 12% or higher, and most preferably at a concentration of about 15%. Concentrations higher than 15% may be utilized in accordance with the invention. However, additional incremental increases in lipophilic emulsifying agent concentration above 15% tend to provide little further incremental benefits in reducing sulfur-associated malodor in sulfur-containing cleanser formulations containing a surfactant, such as an ionic surfactant or a combination of an ionic surfactant with a non nonionic surfactant in such concentration suitable for use as a cleanser. Therefore, concentrations higher than 15% are not preferred.

As used herein, a lipophilic emulsifying agent is an emulsifying agent that has an HLB (hydrophilic-lipophilic balance) value of 10 or lower. The HLB system has a scale of one to 40 and was introduced in Griffin, WC, “Classification of Surface-Active Agents by HLB”, Journal of the Society of Cosmetic Chemists, 1:311 (1949) and in Griffin, WC, “Calculation of HLB Values of Non-Ionic Surfactants”, Journal of the Society of Cosmetic Chemists, 5:259 (1954).

In a preferred embodiment, the formulation further contains a sulfonamide. A preferred sulfonamide is sodium sulfacetamide.

The form of sulfur that is contained in the cleanser includes any organic or inorganic sulfur ingredient that is suitable for use in dermatologic applications. For example, the sulfur may be elemental sulfur, a sulfite, a sulfide, a sulfate, a thiosulfate, or a mercaptan.

Any therapeutically useful concentration of sulfur in the formulation is suitable for the invention. Preferably, the concentration of sulfur is from about 1% to about 25%, typically between about 2% to about 20%. More preferably, the concentration of sulfur is between 3% and 10%. Most preferably, the concentration of sulfur is about 5%. These concentrations of sulfur in the composition refer to the active therapeutic ingredient and are exclusive of any sulfur that is contained in any surfactant, sulfonamide, or other ingredient that may or may not be present in the formulation.

The concentration of surfactant or surfactants in the formulation is sufficiently high to provide a skin-cleansing effective amount of surfactant when the formulation is applied to the skin. Typical ranges of surfactant concentration is 5 to 50%, usually 10 to 35%, and most commonly from 15 to 25%.

Any surfactant or group of surfactants that is suitable for dermatologic applications is suitable for the invention. Thus, the surfactants may be nonionic, anionic, cationic, zwitterionic, amphoteric, or ampholytic surfactants. Such surfactants are well known in the art and are disclosed in Orr et al., U.S. Pat. No. 4,976,953; Ciotti et al., U.S. Pat. No. 5,011,681; and Harmalker et al., U.S. Pat. No. 6,150,313; each of which is incorporated herein by reference.

Preferred surfactants include a combination of disodium laureth sulfosuccinate and sodium lauryl sulfoacetate, such as sold under the trade name STEPAN-MILD LSB™ (Stepan Company, Northfield, Ill.), sodium cocoyl isethionate, such as sold under the trade name JORDAPON™ (BASF Corporation, Mount Olive, N.J.), and polypropylene glycol (PPG)-2-hydroxyethylcoco isostearamide, such as sold under the trade name PROMODIUM 2™ (Uniquema Inc., New Castle, Del.).

A preferred lipophilic emulsifying agent is emulsifying wax. The term “emulsifying wax” indicates one or more of the many solid nonionic emulsifiers that are known in the art and that are prepared as a mixture of fatty acids of about 12 to about 24 carbon atoms in length.

Emulsifying waxes that are preferred are those that meet the standards of the National Formulary (N.F.) or British Pharmacopeia (B.P.). Such waxes may be self-emulsifying or non-self-emulsifying. A preferred N.F. grade emulsifying wax is prepared from cetostearyl alcohol containing a polyoxyethylene derivative of a fatty ester of sorbitan. This material is known as Emulsifying Wax N.F. and is a creamy white, wax-like solid that is freely soluble in ether, chloroform, alcohol, and most hydrocarbon solvents. It is insoluble in water. Emulsifying Wax N.F. is available from several manufactures, for example the emulsifying waxes sold under the trade names POLAWAX™ (Croda, Inc., NY) and LIPOWAX™ (Lipo Chemicals, Inc., Paterson, N.J.).

Any sulfonamide that is suitable for topical administration to the skin may be included in the formulation of the invention. Sulfonamides are synthetic antibacterial agents composed of one or more benzene rings, amino groups, and a sulfonamide group (SO₂NH₂). A preferred sulfonamide is sulfacetamide. The preferred form of sulfacetamide is the salt form, and most preferably the sodium salt. Examples of other suitable sulfonamides include sulfadiazine, phthalylsulfacetamide, phthalylsulfathiazole, succinylsulfathiazole, sulfabenzamide, sulfaethidole, sulfaguanidine, sulfamethizole, sulfamethoxypyridazine, sulfanilamide, sulfanilamidomethanesulfonic acid triethanolamine salt, sulfanitran, sulfapyridine, sulfathiazole, sulfisoxazole, cetyl sulfamethoxypyrazine, N-2-formylsulfisomidine, salazosulfadimidine, sulfachlorpyridazine, sulfadimethoxine, sulfadoxine, sulfalene, sulfamerazine, sulfameter, sulfamethazine, sulfamethomidine, sulfaperine, sulfaphenazole, sulfapyrazine and sulfisomidine.

The concentration of sulfonamide in the formulation of the invention is that which is sufficient to provide a therapeutic benefit, such as an antimicrobial benefit, to the skin. In a preferred embodiment, the sulfonamide is sulfacetamide at a concentration of between 1% and 35%. More preferably, the concentration of sulfacetamide is between 5% and 20%. Most preferably, the concentration of sulfacetamide is about 7.5% to 15%, with a most preferred concentration of about 10%.

Other therapeutic agents, in place of or in addition to a sulfonamide, may be present in the formulation of the invention. Such therapeutic agents may include antimicrobial agents, such as antibiotics or antifungal agents, anti-inflammatory agents such as corticosteroids, immunomodulators or immunosuppressive agents, anti-parasitic agents, keratinization modulating agents, depigmenting agents, analgesic agents, or sunblock agents.

The formulation of the invention may additionally include one or more optional ingredients. Such optional ingredients include dyes and pigments, thickening agents, preservatives, stabilizers such as anti-oxidants, chelating agents, foam boosting and stabilizing agents, and a vehicle such as an organic or inorganic solvent such as water, propylene glycol, or polyethylene glycol. Perfumes, fragrances, and/or masking agents are not preferred as a component of the formulation, especially in view of the fact that the formulation of the invention has decreased sulfur-related malodor compared to prior art sulfur-containing cleansers. However, if desired, a perfume, fragrance, or masking agent may be an ingredient of the formulation. Preferably, the formulation of the invention is perfume and fragrance free, and most preferably is perfume, fragrance, and masking agent free.

Thickening agents are often used in sulfur-containing formulations to reduce the tendency of sulfur to flocculate during preparation or storage, with resultant precipitation and deterioration of the sulfur-containing product. If a thickening agent is utilized, a preferred thickening agent is polypropylene glycol-2 hydroxyethylcoco isosteramide, sold under the brand name PROMIDIUM 2™ (Uniquema, New Castle, Del.). This thickening agent is especially preferred in formulations containing cleansing agents and an active ingredient that is a salt, such as sodium sulfacetamide. With such formulations, thickening agents such as hydroxymethylcellulose, hydroxyethylcellulose, polyvinyl pyrrolidone, or poloxamer are generally not preferred due to the propensity for phase separation or polymer precipitation.

Likewise, common emulsifiers such as polysorbate 60, steareth 20, or triethanolamine stearate are not preferred for the formulation of the invention due to possible phase separation in the presence of high salt concentration, such as when the preferred salt form of a sulfonamide, such as the preferred sodium sulfacetamide, is used. Thus, in a formulation with high salt concentration, such as one containing sodium sulfacetamide, for example at a 10% concentration in addition to high concentrations of surfactants, emulsifying wax is the preferred lipophilic emulsifying agent.

In a cleanser formulation containing sulfur at 5%, one or more surfactants in a concentration typically found in cleansers, and sodium sulfacetamide, a concentration of emulsifying wax of 7% or higher inhibits or prevents phase separation. Especially at high concentrations of emulsifying was, such as at a concentration of 15%, no phase separation is noted, even upon prolonged storage. At these levels of emulsifying wax, sulfur malodor is markedly reduced and at a concentration of 15%, most testers find that virtually no sulfur malodor is present, even in the absence of a masking agent or fragrance.

In a preferred embodiment, the formulation of the invention contains about 5% to about 30% of cleansing agent, about 1% to about 25% of an optional thickening agent, 7% to about 35% of lipophilic emulsifier, about 1% to about 25% sulfur, about 1% to about 35% sodium sulfacetamide, and, if desired, additional therapeutic agents such as keratolytic, antimicrobial, antifungal, antiparasitic or anti-inflammatory agents, and other ingredients such as stabilizers like anti-oxidants, preservatives, and chelating agents.

The formulation of the invention may be made by any of several methods known in the art for making sulfur-containing cleanser formulations. Sulfur, surfactant, and emulsifying agent are combined in a mixture to obtain the formulation. Typically, sulfur is dispersed and homogenized in surfactant and water to obtain a slurry. The sulfur slurry is then added to a mixture containing other ingredients, such as preservatives, chelating agents, antioxidants, cleansing agents, lipophilic emulsifying agent, and a sulfonamide such as sodium sulfacetamide.

In a preferred embodiment, the sodium sulfacetamide and sulfur-containing cleanser formulation of the invention is made by preparing two phases, an aqueous solution and an oil phase. The aqueous phase is made by combining in purified water preservatives such as methylparaben and propylparaben, a chelating agent such as disodium EDTA, an antioxidant such as butylated hydroxytoluene or sodium thiosulfate, and a cleansing agent such as sodium lauryl sulfoacetate & disodium laureth sulfosuccinate or sodium cocoyl isethionate. Other optional ingredients, as described above, may also be combined. This mixture is then heated to about 70° C. The oil phase is prepared by melting emulsifying wax, preferably with light mineral oil at about 70° C. The oil phase is then added to the aqueous mixture with mixing. After a homogeneous mixture of the aqueous and oil phases is obtained, the mixture is cooled to about 40° C. or lower and then sodium sulfacetamide is added to the mixture and mixed until homogenous.

In a separate vessel, a sulfur slurry is made by dispersing sulfur in a thickening surfactant agent, such as PPG-2 hydroxyethyl coco isostearamide and water, and homogenizing the resulting mixture until no aggregates are observed. The sulfur slurry is then combined with the sodium sulfacetamide mixture by mixing to obtain the cleanser formulation.

The cleanser formulation of the invention may be used for the topical treatment of dermatitis, such as acne vulgaris, rosacea, and seborrheic dermatitis, or other skin disorder by washing an affected area of skin one or more times daily with the formulation. Preferably, an individual suffering from such disorder wets the affected area of skin that is to be cleansed, applies the formulation to the area, massages the formulation into the skin for several seconds, such as up to 10 or 20 seconds or more, works the formulation into a lather, and then rinses the skin thoroughly to remove the formulation. The invention may be used as a shampoo for the treatment of scalp conditions, such as seborrheic dermatitis, while cleansing the hair and scalp.

The invention is further illustrated in the following non-limiting examples.

### EXAMPLE 1

### Preferred Cleanser of the Invention

A cleanser formulation of the invention was prepared according to the preferred method described above. Table I shows the ingredients with concentration ranges of a preferred skin cleanser formulation according to the invention.

TABLE I
Quantitative Composition of a Preferred Cleanser of the Invention

| COMPONENT | % (w/w) |
|---|---|
| sulfur | 1 to 25 |
| methylparaben | 0.1 to 0.25 |
| propylparaben | 0.01 to 0.04 |
| sodium cocoyl isethionate | 3 to 10 |
| sodium lauryl sulfoacetate | 2.5 to 15 |
| & disodium laureth | |
| sulfosuccinate (STEPAN- | |
| MILD LSB ™) | |
| PPG-2 hydroxyethyl coco/ | 1 to 5 |
| isostearamide | |
| lipophilic emulsifying | 7 to 35 |
| agent, i.e. emulsifying wax | |
| light mineral oil | 0 to 1 |
| purified water q.s.ad | 100 |

### EXAMPLE 2

### Preferred Sulfonamide-Containing Cleanser of the Invention

A cleanser formulation of the invention containing a sulfonamide was prepared according to the preferred method described above. Table 2 shows the ingredients with concentration ranges of a preferred sulfonamide-containing skin cleanser formulation according to the invention.

TABLE 2
Quantitative Composition of a Preferred Cleanser of the Invention

| COMPONENT | % (w/w) |
|---|---|
| sodium sulfacetamide | 1 to 35 |
| sulfur | 1 to 25 |
| methylparaben | 0.1 to 0.25 |
| propylparaben | 0.01 to 0.04 |
| disodium EDTA | 0.01 to 0.25 |
| butylated hydroxytoluene | 0.01 to 0.2 |
| sodium thiosulfate | 0.05 to 0.5 |
| sodium cocoyl isethionate | 3 to 10 |
| sodium lauryl sulfoacetate | 2.5 to 15 |
| & disodium laureth | |
| sulfosuccinate (STEPAN- | |
| MILD LSB ™) | |
| PPG-2 hydroxyethyl coco/ | 1 to 5 |
| isostearamide | |
| diluted hydrochloric acid 0.25 to 1.5 | |
| lipophilic emulsifying | 7 to 35 |
| agent, i.e. emulsifying wax | |
| light mineral oil | 0 to 1 |
| purified water q.s.ad | 100 |

### EXAMPLE 3

### Preferred Shampoo of the Invention

A preferred shampoo formulation of the invention was prepared with the ingredients and concentrations shown in Table 3.

TABLE 3
Quantitative Composition of a Preferred Shampoo of the Invention

| COMPONENT | % (w/w) |
|---|---|
| sulfur | 1 to 25 |
| benzyl alcohol | 0.5 to 2.5 |
| sodium cocoyl isethionate | 3 to 10 |
| sodium laureth sulfate, 30% | 2.5 to 15 |
| sodium lauryl sulfoacetate | 2.5 to 20 |
| & disodium laureth | |
| sulfosuccinate | |
| PPG-2 hydroxyethyl coco/ | 1 to 8 |
| isostearamide | |
| lipophilic emulsifying | 7 to 35 |
| agent, i.e. emulsifying wax | |
| purified water q.s.ad | 100 |

### EXAMPLE 4

### Preferred Sulfonamide-Containing Shampoo of the Invention

A preferred sulfonamide-containing shampoo formulation of to the invention was prepared with the ingredients and concentrations shown in Table 4.

TABLE 4
Quantitative Composition of a Preferred Shampoo of the Invention

| COMPONENT | % (w/w) |
|---|---|
| sodium sulfacetamide | 1 to 35 |
| sulfur | 1 to 25 |
| methylparaben | 0.1 to 0.25 |
| propylparaben | 0.01 to 0.04 |
| benzyl alcohol | 0.0 to 2.0 |
| disodium EDTA | 0.01 to 0.25 |
| butylated hydroxytoluene | 0.01 to 0.2 |
| sodium thiosulfate | 0.05 to 0.5 |
| sodium cocoyl isethionate | 3 to 10 |
| sodium lauryl sulfoacetate | 2.5 to 15 |
| & disodium laureth | |
| sulfosuccinate | |
| PPG-2 hydroxyethyl coco/ | 1 to 5 |
| isostearamide | |
| diluted hydrochloric acid | 0.25 to 1.5 |
| lipophilic emulsifying | 7 to 35 |
| agent, i.e. emulsifying wax | |
| light mineral oil | 0 to 1 |
| purified water q.s.ad | 100 |

The shampoo of Example 4 was prepared by the following illustrative procedure. Heat a mixture, preferably a solution, of purified water, methylparaben, propylparaben, disodium EDTA, butylated hydroxytoluene, sodium thiosulfate, sodium cocoyl isethionate, sodium lauryl sulfoacetate & sodium sulfosuccinate, and diluted hydrochloric acid to 70 to 80° C. Add an oil phase containing emulsifying wax, light mineral oil, and benzyl alcohol that is at a temperature of 70 to 80° C. Mix and allow to cool to a temperature of about 40° C. Add sodium sulfacetamide and mix until uniform. Add a mixture of sulfur and PPG-2 hydroxyethyl coco/isostearamide that has been predispersed by a homogenizing mixer and mix until homogenous while allowing to cool to room temperature.

### EXAMPLE 5

### Comparison of Residual Sulfur Odor After Using a Skin Cleanser of the Invention Compared to the Closest Prior Art

A double blind test was performed to compare a commercially available skin cleanser according to the invention (ROSANIL™ Cleanser, Galderma Laboratories, LP, Fort Worth, Tex.) and a commercially available skin cleanser of the prior art (PLEXION™ Cleanser, Medicis, Scottsdale, Ariz.). Each of these two skin cleansers contains 10% sodium sulfacetamide and 5% sulfur.

ROSANIIL further contains butylated hydroxytoluene, edetate sodium, emulsifying wax, hydrochloric acid, light mineral oil, methylparaben, PPG-2 hydroxyethyl coco/isostearamide, propylparaben, purified water, sodium cocoyl isethionate, sodium lauryl sulfoacetate and disodium laureth sulfosuccinate, and sodium thiosulfate. According to the package insert, PLEXION further contains water, sodium methyl oleyltaurate, sodium cocoyl isethionate, disodium oleamide MEA sulfosuccinate, cetyl alcohol NF, glyceryl stearate and PEG-100 stearate, stearyl alcohol NF, PEG-55 propylene glycol oleate, magnesium aluminum silicate NF, methylparaben NF, disodium EDTA, butylated hydroxytoluene NF, sodium thiosulfate, fragrance, xantham gum NF, and propylparaben NF.

Six subjects participated in the double-blind study. The study lasted 3 days. On day 1 of the study, each of the subjects used one of the two cleansers to cleanse the face as directed on the product labeling. Each subject washed his or her face with either the ROSANIL cleanser of the invention or the PLEXION cleanser of the prior art by wetting the skin and liberally applying the cleanser to the skin, then gently massaging the cleanser into the skin for 10 to 20 seconds to work the cleanser into a full lather. Then, the cleanser was rinsed off and the skin was patted dry.

Day 2 of the study was a wash-out period during which no test material was used. On that day 2, each participant washed his or her face using a cleanser that did not contain sulfur. On day 3 of the study, each subject washed his or her face as on day 1 but each subject used the test cleanser that he or she did not use on day 1 of the study.

On days 1 and 3, each subject completed a questionnaire in which the subject assessed (a) malodor during use, (b) residual malodor during the first hour following washing, and (c) residual malodor during the next four (4) hours following washing. Additionally, following day 3, each subject was questioned regarding his or her preference between the two test products and each of the product's suitability for daily use as a facial cleanser. The results of the study concerning malodor are summarized in Table 5.

TABLE 5

| | Rosanil | | Plexion | |
|---|---|---|---|---|
| | (product of the invention) | | (product of the prior art) | |
| | number | number | number | number |
| | reporting no | reporting | reporting no | reporting |
| | unpleasant | unpleasant | unpleasant | unpleasant |
| | sulfur odor | sulfur odor | sulfur odor | sulfur odor |
| While washing | 4 | 2 | 1 | 5 |
| face | | | | |
| During 1st hour | 4 | 2 | 2 | 4 |
| following face | | | | |
| washing | | | | |
| During 4 hours | 5 | 1 | 4 | 2 |
| following face | | | | |
| washing | | | | |

The results shown in Table 5 establish that the test subjects experienced markedly less sulfur malodor when using the product of the invention compared to the closest prior art.

This reduction in malodor was present at all three time periods tested; during washing, within the first hour following washing, and within 4 hours following washing. During washing, 67% of subjects reported no malodor with Rosanil and only 17% reported no malodor with Plexion.

During the first hour, 67% of subjects reported no malodor with Rosanil and only 33% reported no malodor with Plexion. During the four hour period following washing 83% of subjects reported no malodor with Rosanil compared to 67% of subjects that reported no malodor with Plexion. These results are especially striking because the Rosanil product of the invention contains no sulfur malodor masking fragrance, in contrast with the Plexion product of the prior art which does contain such sulfur malodor masking fragrance.

When questioned following the study, 100% of the subjects said that they would use the Rosanil product of the invention as a daily medicated facial cleanser, whereas only 67% of the subjects said that they would use the Plexion product of the prior art as a daily medicated facial cleanser. When questioned regarding their preferences as to either of the two cleansers, 100% of the subjects having a preference preferred the Rosanil product over the Plexion product. Five of the six subjects (83%) stated that they preferred Rosanil to Plexion. One of the six subjects (17%) indicated no preference. None of the subjects preferred the Plexion cleanser over the Rosanil cleanser.

While the foregoing has presented specific embodiments of the present invention, it is to be understood that these embodiments have been presented by way of example only. It is expected that others skilled in the art will perceive variations which, while varying from the foregoing, do not depart from the spirit and scope of the invention herein described and claimed. None of the foregoing is attempted in any manner to limit the scope of the present invention. It is intended that such variations are included within the scope of the following claims.

1. A cleanser formulation comprising sulfur, a surfactant, and a lipophilic emulsifying agent, wherein the concentration of the emulsifying agent in the formulation is 7% w/w or higher.

2. The cleanser formulation of claim 1 wherein the formulation has decreased malodor compared with a similar second cleanser formulation having the same concentration of sulfur and surfactant but having a concentration of the emulsifying agent less than 7% when the cleansers are applied to skin.

3. The cleanser formulation of claim 1 wherein the lipophilic emulsifying agent is emulsifying wax.

4. The cleanser formulation of claim 3 wherein the emulsifying wax is Emulsifying Wax N.F.

5. The cleanser formulation of claim 1 wherein the concentration of emulsifying agent is between 8% and 20%.

6. The cleanser formulation of claim 1 wherein the concentration of emulsifying agent is between 10% and 15%.

7. The cleanser formulation of claim 1 wherein the concentration of emulsifying agent is about 15%.

8. The cleanser formulation of claim 1 wherein the concentration of emulsifying agent is over 15%.

9. The cleanser formulation of claim 1 wherein the surfactant is selected from the group consisting of nonionic, anionic, cationic, zwitterionic, amphoteric, and ampholytic surfactant.

10. The cleanser formulation of claim 1 wherein the surfactant is an ionic surfactant or a combination of ionic and non-ionic surfactants.

11. The cleanser formulation of claim 10 wherein the surfactant is one or more surfactants selected from the group consisting of sodium cocoyl isethionate, polypropylene glycol (PPG)-2-hydroxyethylcoco isostearamide, and a combination of disodium laureth sulfosuccinate and sodium lauryl sulfoacetate.

12. The cleanser formulation of claim 1 wherein the concentration of surfactant in the formulation is about 5 to about 50%.

13. The cleanser formulation of claim 12 wherein the concentration of surfactant in the formulation is 10 to 35%.

14. The cleanser formulation of claim 13 wherein the concentration of surfactant in the formulation is 15 to 25%.

15. The cleanser formulation of claim 1 wherein the sulfur is selected from the group consisting of elemental sulfur, a sulfite, a sulfide, a sulfate, a thiosulfate, and a mercaptan.

16. The cleanser formulation of claim 1 wherein the concentration of sulfur in the formulation is between about 1% and about 25%.

17. The cleanser formulation of claim 16 wherein the concentration of sulfur is between 2% and 20%.

18. The cleanser formulation of claim 17 wherein the concentration of sulfur is between 3% and 10%.

19. The cleanser formulation of claim 18 wherein the concentration of sulfur is about 5%.

20. The cleanser formulation of claim 1 which further comprises a sulfonamide.

21. The cleanser formulation of claim 20 wherein the sulfonamide is sulfacetamide.

22. The cleanser formulation of claim 21 wherein the concentration of sulfacetamide in the cleanser is between about 1% and about 35%.

23. The cleanser formulation of claim 22 wherein the concentration of sulfacetamide is between 5% and 20%.

24. The cleanser formulation of claim 23 wherein the concentration of sulfacetamide is between 7.5% and 15%.

25. The cleanser formulation of claim 24 wherein the concentration of sulfacetamide is about 10%.

26. The cleanser formulation of claim 25 wherein the concentration of sulfur in the formulation is about 5%.

27. The cleanser formulation of claim 1 which is free of perfumes and fragrances.

28. The cleanser formulation of claim 27 which is free of masking agents.

29. The cleanser formulation of claim 1 which comprises the following ingredients in the following concentrations w/w: 5% to 30% surfactant, 7% to 35% lipophilic emulsifier, 2% to 20% sulfur, and 5% to 20% sodium sulfacetamide.

30. The cleanser formulation of claim 29 which further comprises 1 to 25% thickening agent.

31. The cleanser of claim 1 which comprises the following ingredients: sulfur, methylparaben, propylparaben, sodium cocoyl isethionate, sodium lauryl sulfoacetate, disodium laureth sulfosuccinate, polypropylene glycol-2 hydroxyethyl coco isostearamide, emulsifying wax, and 0 to 1% mineral oil, wherein the concentration of emulsifying wax is at least 7%.

32. The cleanser formulation of claim 31 which comprises the following ingredients in the following concentrations w/w: 1 to 25% sulfur, 0.1 to 0.25% methylparaben, 0.01 to 0.04% propylparaben, 3 to 10% sodium cocoyl isethionate, 2.5 to 15% combination of sodium lauryl sulfoacetate and disodium laureth sulfosuccinate, 1 to 5% polypropylene glycol-2 hydroxyethyl coco isostearamide, 7 to 35% emulsifying wax, and 0% or more mineral oil.

33. The cleanser formulation of claim 1 which comprises the following ingredients: sodium sulfacetamide, sulfur, methylparaben, propylparaben, disodium EDTA, butylated hydroxytoluene, sodium thiosulfate, sodium cocoyl isethionate, sodium lauryl sulfoacetate, disodium laureth sulfosuccinate, polypropylene glycol-2 hydroxyethyl coco isostearamide, diluted hydrochloric acid, emulsifying wax, and 0% or more mineral oil.

34. The cleanser formulation of claim 33 which comprises the following ingredients in the following concentrations w/w: 5 to 15% sodium sulfacetamide, 2 to 10% sulfur, 0.1 to 0.25% methylparaben, 0.01 to 0.04% propylparaben, 0.01 to 0.25% disodium EDTA, 0.01 to 0.2% butylated hydroxytoluene, 0.05 to 0.5% sodium thiosulfate, 3 to 10% sodium cocoyl isethionate, 2.5 to 15% combination of sodium lauryl sulfoacetate and disodium laureth sulfosuccinate, 1 to 5% polypropylene glycol-2 hydroxyethyl coco isostearamide, 0.25 to 1.5% diluted hydrochloric acid, 7 to 35% emulsifying wax, and 0% or more mineral oil.

35. A method for making a cleanser formulation comprising combining sulfur, a surfactant, and a lipophilic emulsifying agent to obtain the formulation, wherein the concentration of the lipophilic emulsifying agent in the formulation is at least about 7% w/w.

36. The method of claim 35 wherein the sulfur is dispersed and homogenized with a surfactant and water to form a slurry and the slurry is combined with other ingredients including the lipophilic emulsifying agent to obtain the formulation.

37. The method of claim 35 wherein the lipophilic emulsifying agent is emulsifying wax.

38. The method of claim 37 wherein the emulsifying wax is Emulsifying Wax N.F.

39. The method of claim 35 wherein the concentration of emulsifying agent is between 10% and 15%.

40. The method of claim 35 wherein the concentration of emulsifying agent is about 15%.

41. The method of claim 35 wherein the concentration of emulsifying agent is over 15%.

42. The method of claim 35 wherein the surfactant is selected from the group consisting of nonionic, anionic, cationic, zwitterionic, amphoteric, and ampholytic surfactants.

43. The method of claim 35 wherein the surfactant is an ionic surfactant or a combination of ionic and non-ionic surfactants.

44. The method of claim 43 wherein the surfactant is one or more surfactants selected from the group consisting of sodium cocoyl isethionate, polypropylene glycol (PPG)-2-hydroxyethylcoco isostearamide, and a combination of di sodium laureth sulfosuccinate and sodium lauryl sulfoacetate.

45. The method of claim 35 wherein the concentration of surfactant in the formulation is about 5 to about 50%.

46. The method of claim 45 wherein the concentration of surfactant in the formulation is 10 to 35%.

47. The method of claim 46 wherein the concentration of surfactant in the formulation is 15 to 25%.

48. The method of claim 35 wherein the sulfur is selected from the group consisting of elemental sulfur, a sulfite, a sulfide, a sulfate, a thiosulfate, and a mercaptan.

49. The method of claim 35 wherein the concentration of sulfur in the formulation is between 1% and 25%.

50. The method of claim 49 wherein the concentration of sulfur is between 2% and 20%.

51. The method of claim 50 wherein the concentration of sulfur is between 3% and 10%.

52. The method of claim 51 wherein the concentration of sulfur is about 5%.

53. The method of claim 35 which further comprises combining a sulfonamide with the sulfur, the surfactant, and the lipophilic emulsifying agent.

54. The method of claim 53 wherein the sulfonamide is sulfacetamide.

55. The method of claim 54 wherein the concentration of sulfacetamide in the formation is between 1% and 35%.

56. The method of claim 55 wherein the concentration of sulfacetamide is between 5% and 20%.

57. The method of claim 56 wherein the concentration of sulfacetamide is between 7.5% and 15%.

58. The method of claim 57 wherein the concentration of sulfacetamide is about 10%.

59. The method of claim 58 wherein the concentration of sulfur in the formulation is about 5%.

60. The method of claim 35 wherein no perfumes or fragrances are combined in the formulation.

61. The method of claim 60 wherein no masking agents are combined in the formulation.

62. The method of claim 35 wherein the following ingredients in the following concentrations w/w are combined to obtain the formulation: about 5% to about 30% surfactant, about 1% to about 25% thickening agent, about 7% to about 35% lipophilic emulsifier, about 2% to about 20% sulfur, and about 5% to about 20% sodium sulfacetamide.

63. The method of claim 35 wherein the following ingredients are combined to obtain the formulation: sulfur, methylparaben, propylparaben, sodium cocoyl isethionate, sodium lauryl sulfoacetate, disodium laureth sulfosuccinate, polypropylene glycol-2 hydroxyethyl coco isostearamide, and emulsifying wax, wherein the concentration of emulsifying wax in the formulation is between 7% and 35%.

64. The method of claim 63 wherein the following ingredients in the following concentrations w/w are combined to obtain the formulation: 1 to 25% sulfur, 0.1 to 0.25% methylparaben, 0.01 to 0.04% propylparaben, 3 to 10% sodium cocoyl isethionate, 2.5 to 15% combination of sodium lauryl sulfoacetate and disodium laureth sulfosuccinate, 1 to 5% polypropylene glycol-2 hydroxyethyl coco isostearamide, 7 to 35% emulsifying wax, and 0% or more mineral oil.

65. The method of claim 35 wherein the following ingredients are combined to obtain the formulation: sodium sulfacetamide, sulfur, methylparaben, propylparaben, disodium EDTA, butylated hydroxytoluene, sodium thiosulfate, sodium cocoyl isethionate, sodium lauryl sulfoacetate, disodium laureth sulfosuccinate, polypropylene glycol-2 hydroxyethyl coco isostearamide, diluted hydrochloric acid, and emulsifying wax, wherein the concentration of emulsifying wax in the formulation is between 7 and 35%.

66. The method of claim 65 wherein the following ingredients in the following concentrations w/w are combined to obtain the formulation: 5 to 15% sodium sulfacetamide, 2 to 10% sulfur, 0.1 to 0.25% methylparaben, 0.01 to 0.04% propylparaben, 0.01 to 0.25% disodium EDTA, 0.01 to 0.2% butylated hydroxytoluene, 0.05 to 0.5% sodium thiosulfate, 3 to 10% sodium cocoyl isethionate, 2.5 to 15% combination of sodium lauryl sulfoacetate and disodium laureth sulfosuccinate, 1 to 5% polypropylene glycol-2 hydroxyethyl coco isostearamide, 0.25 to 1.5% diluted hydrochloric acid, 7 to 35% emulsifying wax, and 0% or more mineral oil.

67 A cleanser formulation made by the method of claim 35.

68. A cleanser formulation made by the method of claim 62.

69. A cleanser formulation made by the method of claim 63.

70. A cleanser formulation made by the method of claim 64.

71. A cleanser formulation made by the method of claim 65.

72. A cleanser formulation made by the method of claim 66.

73. A cleanser formulation comprising sulfur, a surfactant, and emulsifying wax, wherein the concentration of the emulsifying wax in the formulation is 7% w/w or higher.

74. The cleanser formulation of claim 73 wherein the emulsifying wax is Emulsifying Wax N.F.

75. The cleanser formulation of claim 73 wherein the concentration of the emulsifying wax is between 8% and 20%.

76. The cleanser formulation of claim 73 wherein the concentration of emulsifying wax is between 10% and 15%.

77. The cleanser formulation of claim 73 wherein the concentration of emulsifying wax is over 15%.

79. The cleanswer formulation of claim 73 wherein the sulfur is selected from the group consisting of elemental sulfur, a sulfite, a slufide, a sulfate, a thiosulfate, and a mercaptan.

80. The cleanser formulation of claim 73 wherein the concentration of sulfur in the formulation if between about 1% and about 25%.

81. The cleanser formulation of claim 73 which further comprises a sulfonamide.

82. The cleanser formulation of claim 81 wherein the sulfonamide is sulfacetamide.

83. The cleanser formulation of claim 82 wherein the concentration of sulfacetamide in the cleanser is between about 1% and about 35%.

84. The cleanser formulation of claim 83 wherein the concentration of sulfacetamide is between 5% and 20%.

85. The cleanser formulation of claim 84 wherein the concentration of sulfacetamide is between 7.5% and 15%.

86. The cleanser formulation of claim 85 wherein the concentration of sulfacetamide is about 10%.

87. The cleanser formulation of claim 86 wherein the concentration of sulfur in the formulation is about 5%.

88. The cleanser formulation of claim 73 which is free of perfumes and fragrances.

89. The cleanser formulation of claim 88 which is free of masking agents.

90. The cleanser formulation of claim 73 which comprises the following ingredients in the following concentrations w/w: 5% to 30% surfactant, 7% to 35% emulsifying wax, 2% to 20% sulfur, and 5% to 20% sodium sulfacetamide.
